(19) United States
(12) Patent Application Publication
(10) Pub. No.: US 2026/0144982 A1
(43) Pub. Date: May 28, 2026

(54) WEARABLE NEURAL STIMULATION AND SENSING DEVICE

(71) Applicant: National Yang Ming Chiao Tung University, Hsinchu City (TW)

(72) Inventors: Yu-Te Liao, Hsinchu City (TW); Lin Chou, Tainan City (TW); Yi-Jie Lin, Kaohsiung City (TW); Po-Hsun Chu, Taipei City (TW); Hai-Yin Chen, Tainan City (TW)

(73) Assignee: National Yang Ming Chiao Tung University, Hsinchu City (TW)

(21) Appl. No.: 19/259,209

(22) Filed: Jul. 3, 2025

(30) Foreign Application Priority Data
Nov. 22, 2024 (TW) 113145056

(51) Int. Cl. A61N 1/04; A61N 1/36

(52) U.S. Cl. CPC A61N 1/0484; A61N 1/36031

(57) ABSTRACT

A wearable neural stimulation and sensing device includes a headband worn on a head. The surface of the headband has electrode mounting recesses that can flexibly mount a stimulation electrode and a sensing electrode. The stimulation electrode includes a shallow stimulation electrode and a deep stimulation electrode that can respectively perform trigeminal nerve stimulation (TNS) and temporal interference brain stimulation (TIBS) to implement non-invasive electrical stimulation for shallow and deep brain regions. The sensing electrode, used for electrical impedance tomography, monitors the physiological data of brain activity in real time. A control unit adjusts the electrical stimulation parameter of the stimulation electrode and the measurement parameter of the sensing electrode. A processing unit processes the obtained physiological data. The present invention features precise stimulation, compact size, and portability, making it suitable for the treatment and monitoring of neurodegenerative diseases, thereby improving the quality of life of patients.

### BACKGROUND OF THE INVENTION

This application claims priority for the TW patent application No. 113145056 filed on 22 Nov. 2024, the content of which is incorporated by reference in its entirely.

### FIELD OF THE INVENTION

The invention relates to a technical field for wearable medical devices, particularly to a wearable neural stimulation and sensing device.

### DESCRIPTION OF THE RELATED ART

Currently, treatments for neurodegenerative diseases mainly depend on invasive electrical stimulation, pharmacological therapies, and assistance from large-scale medical equipment. Invasive electrical stimulation, such as Deep Brain Stimulation (DBS), requires electrodes to be implanted in the deep brain regions of patients. Although the method is effective in some cases, the method involves high surgical risks and costs and may lead to complications such as infection and bleeding, placing a significant physical burden on patients.

Non-invasive stimulation techniques, such as Transcranial Magnetic Stimulation (TMS) and Transcranial Direct Current Stimulation (tDCS), have been applied in the treatment of some neurodegenerative diseases. However, these techniques typically require large and stationary equipment, meaning patients must travel to medical institutions for treatment, making portable or home-based therapy unfeasible. Furthermore, since the stimulation depth of these techniques is limited, the techniques are less effective in targeting deep brain regions, thereby reducing treatment precision and efficacy.

In terms of diagnostic imaging, traditional devices such as Magnetic Resonance Imaging (MRI) and Computed Tomography (CT) scanners are bulky and costly, making them unsuitable for daily monitoring or portable use. These systems also fail to reflect real-time brain activity, limiting clinicians' ability to evaluate and adjust treatment plans promptly.

Monitoring physiological signals typically requires specialized medical devices, such as electrocardiographs or electroencephalographs, which are also large and complex to operate, making them difficult for patients to use independently in daily life. Additionally, existing technologies often use electrical stimulation devices and physiological signal monitoring equipment as independent systems that lack integration, thereby increasing user inconvenience.

In summary, the existing technologies for treating and monitoring neurodegenerative diseases face the following challenges: (1) High risk of invasive treatments: Surgical implantation of electrodes increases the risk of surgery and potential complications. (2) Bulky equipment: Non-invasive stimulation and imaging devices are large and non-portable, limiting treatment flexibility and convenience. (3) Limited stimulation depth: Existing non-invasive technologies are insufficient for stimulating deep brain regions, reducing effectiveness for certain conditions. (4) Lack of real-time monitoring: Inability to monitor brain activity and electrical physiological signals in real time hinders real-time treatment assessment and timely treatment adjustments. (5) Low integration: Electrical stimulation devices and electrical physiological signal monitoring devices operate as separate systems, adding complexity in use and reducing user-friendliness.

Therefore, there is an urgent need for a device that addresses the foregoing problems. Such a device will improve treatment efficacy for neurodegenerative diseases, reduce dependence on large medical equipment, and enhance patient convenience and treatment adherence in daily life.

### SUMMARY OF THE INVENTION

The objective of the present invention is to provide a wearable neural stimulation and sensing device, which uses a headband with electrode mounting recesses that flexibly install a stimulation electrode and a sensing electrode. Using the stimulation electrode on the headband, the device can perform non-invasive electrical stimulation on at least one brain region, including deep brain regions and shallow brain regions. A control unit can adjust the electrical stimulation parameter of the stimulation electrode and the measurement parameter of the sensing electrode to precisely stimulating different brain regions and monitoring electrical physiological signals in real time. A processing unit is responsible for processing the electrical physiological signals obtained from the sensing electrode, such as electrical impedance tomography (EIT), electroencephalogram (EEG), electrocardiography (ECG), photoplethysmography (PPG) and galvanic skin response (GSR).

In order to achieve the foregoing purposes, a wearable neural stimulation and sensing device includes a headband, a plurality of electrodes, and a control unit. The headband is worn on the head of a user. The surface of the headband has electrode mounting recesses. The electrodes, mounted in the electrode mounting recesses, include at least two stimulation electrodes. The locations of the stimulation electrodes on the headband correspond to at least one brain region. The control unit is coupled to at least one stimulation electrode and a sensing electrode and configured to adjust the electrical stimulation parameter of at least one stimulation electrode and the measurement parameter of at least one sensing electrode.

The present invention also provides a wearable neural stimulation and sensing device that includes a headband, a plurality of electrodes, a control unit, and a processing unit. The headband is worn on the head of a user. The surface of the headband has electrode mounting recesses. The electrodes, mounted in the electrode mounting recesses, include at least one stimulation electrode and at least one sensing electrode. The stimulation electrode is positioned on the headband at a location corresponding to at least one brain region. The control unit is coupled to the stimulation electrode and the sensing electrode and configured to adjust the electrical stimulation parameter of the stimulation electrode and the measurement parameter of the sensing electrode. The processing unit is configured to obtain an electrical physiological signal from the sensing electrode.

The present invention further provides a wearable neural stimulation and sensing device that includes a headband, a plurality of electrodes, a control unit, and a processing unit. The headband is worn on the head of a user. The surface of the headband has electrode mounting recesses. The electrodes, mounted in the electrode mounting recesses, include at least one stimulation electrode and at least one sensing electrode. The stimulation electrode is positioned on the headband at a location corresponding to at least one brain region. The sensing electrode is configured as an electrical impedance tomography (EIT) electrode. The control unit is coupled to the stimulation electrode and the sensing electrode and configured to adjust the electrical stimulation parameter of the stimulation electrode and the measurement parameter of the sensing electrode. The processing unit is coupled to the sensing electrode and configured to obtain an electrical physiological signal from the sensing electrode and is configured to obtain an intracranial impedance distribution from the EIT electrode to generate an intracranial impedance distribution image.

In an embodiment of the invention, the at least one stimulation electrode includes a shallow stimulation electrode configured to perform the electrical stimulation of a shallow brain region and a deep stimulation electrode configured to perform the electrical stimulation of a deep brain region.

In an embodiment of the invention, the shallow stimulation electrode is a trigeminal nerve stimulation (TNS) electrode. Furthermore, the electrode mounting recesses are arranged on the headband corresponding to the forehead region of the head of the user and configured to mount the TNS electrode to stimulate a trigeminal nerve.

In an embodiment of the invention, the electrical stimulation parameter of the TNS electrode includes a frequency range of 100 Hz˜20 kHz, a current intensity of 0.1˜14 mA, and an interface impedance of 1˜100 kΩ.

In an embodiment of the invention, the deep stimulation electrode is configured as a temporal interference brain stimulation (TIBS) electrode. Furthermore, the electrode mounting recesses are arranged on the headband corresponding to two opposite sides of the head of the user and configured to mount the TIBS electrode to stimulate the deep brain region.

In an embodiment of the invention, the electrical stimulation parameter of the TIBS electrode includes a frequency range of less than 10 kHz, a current intensity of 0.1˜7 mA, and an interface impedance of 5˜20 kΩ.

In an embodiment of the invention, the sensing electrode is configured as an electrical impedance tomography (EIT) electrode. The processing unit obtains an intracranial impedance distribution from the EIT electrode to generate an intracranial impedance distribution image.

In an embodiment of the invention, the measurement parameter of the EIT electrode includes a frequency range of 10 kHz˜100 kHz, a current intensity of 0.1˜1.6 mA, and an interface impedance of 0.1˜10 kΩ.

In an embodiment of the invention, the sensing electrode is configured as an electrical physiological signal sensing electrode, arranged near the postauricular region of the user, and configured to sense electrical physiological signals related to one or a combination of a heart, blood vessels, and skin. The processing unit is coupled to an electrical physiological signal sensor and configured to obtain electrical physiological signals, thereby generating a corresponding electrocardiogram (ECG), photoplethysmogram (PPG), or galvanic skin response (GSR).

The control unit and the processing unit are integrated into a miniaturized chip, which includes an electrical stimulation circuit, an electrical impedance tomography reading circuit, and an electrical physiological electrical signal reading circuit. The electrical stimulation circuit is configured to control the electrical stimulation parameters of the stimulation electrodes. The electrical impedance tomography reading circuit is configured to measure electrical impedance tomography and adjust the measurement parameters. The electrical physiological signal reading circuit is configured to process the physiological signals obtained from the sensing electrodes, including EEG, ECG, PPG, and GSR. Furthermore, the miniaturized chip is configured to avoid signal interference between different currents and achieve precise stimulation, real-time monitoring, and a device design with low power consumption and high efficiency.

Compared to the conventional technology, the invention can achieve the following effects:
(1) Precise Stimulation and High Integration: Electrical stimulation is used to directly modulate abnormal neuronal discharges. The device can be easily integrated with control and sensing circuits into a chip, enhancing the overall device integration.
(2) Integration of Multiple Electrical Stimulation Modalities: Deep and shallow electrical stimulation devices are integrated into a single device, enabling non-invasive stimulation of both deep and shallow brain regions to enhance therapeutic effectiveness.
(3) Integration of Electrical Stimulation and Electrical Impedance Tomography (EIT): The device successfully combines electrical stimulation with EIT technology. High-specification current stimulation circuits prevent signal interference, enabling precise stimulation and real-time monitoring.
(4) High-Functionality Miniaturized Chip: Multiple functional circuits, including electrical stimulation, EIT sensing, and EEG/ECG/PPG/GSR reading circuits, are integrated into a miniaturized chip to achieve a device design with low-power and high performance.
(5) Flexible Adjustment of Clinical Parameter: The device can flexibly change the locations of electrodes and adjust parameters such as stimulation intensity to satisfy different patients' requirements, thereby enabling personalized treatment solutions.
(6) Compact and Portable Wearable Design: The headband-style device is compact, lightweight, and easy to wear, making it suitable for long-term therapy and improving both life quality and treatment compliance of patients.

Below, the embodiments are described in detail in cooperation with the drawings to make easily understood the technical contents, characteristics and accomplishments of the invention.

### BRIEF DESCRIPTION OF THE DRAWINGS

FIG. 1 is a diagram schematically illustrating the state of using a wearable neural stimulation and sensing device according to an embodiment of the invention;

FIG. 2 is a diagram schematically illustrating a wearable neural stimulation and sensing device according to a first embodiment of the invention;

FIG. 3 is a diagram schematically illustrating arrangement of the electrodes of a wearable neural stimulation and sensing device according to an embodiment of the invention; and

FIG. 4 is a diagram schematically illustrating a wearable neural stimulation and sensing device according to a second embodiment of the invention.

### DETAILED DESCRIPTION OF THE INVENTION

Reference will now be made in detail to embodiments illustrated in the accompanying drawings. Wherever possible, the same reference numbers are used in the drawings and the description to refer to the same or like parts. In the drawings, the shape and thickness may be exaggerated for clarity and convenience. This description will be directed in particular to elements forming part of, or cooperating more directly with, methods and apparatus in accordance with the present disclosure. It is to be understood that elements not specifically shown or described may take various forms well known to those skilled in the art. Many alternatives and modifications will be apparent to those skilled in the art, once informed by the present disclosure.

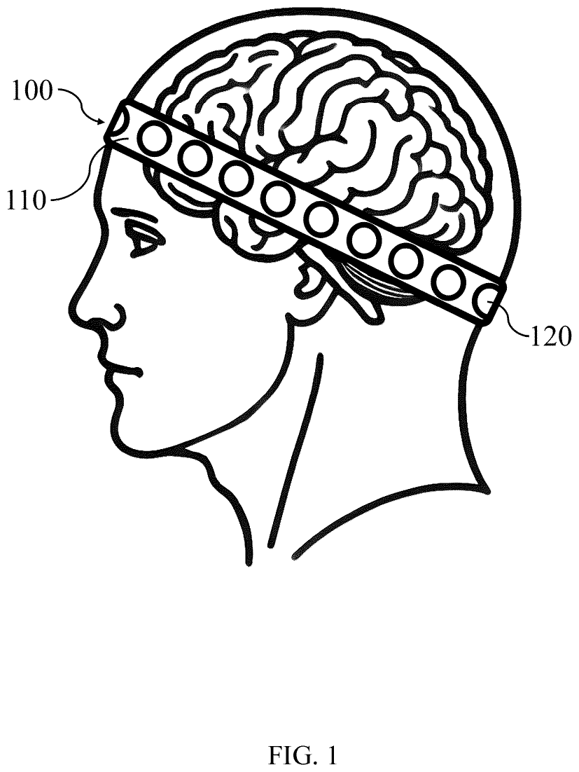

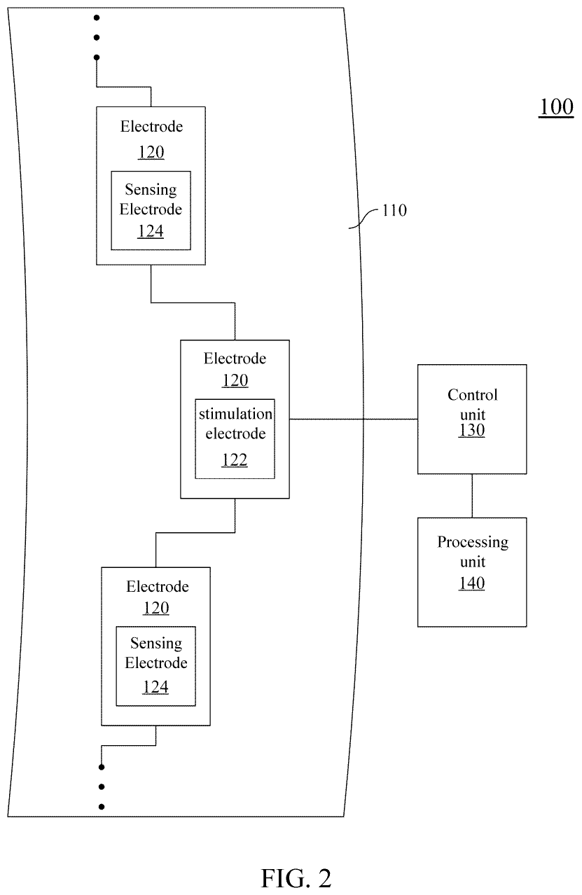

Please refer to FIGS. 1-2. FIG. 1 is a diagram schematically illustrating the state of using a wearable neural stimulation and sensing device according to an embodiment of the invention. FIG. 2 is a diagram schematically illustrating a wearable neural stimulation and sensing device according to a first embodiment of the invention. The order of arranging electrodes 120 in FIG. 2 is for illustration only. The types of electrodes 120 actually installed (e.g., stimulation electrodes 122 or sensing electrodes 124) may be adaptable according to actual requirements.

According to an embodiment, a wearable neural stimulation and sensing device 100 is provided, which includes a headband 110, a plurality of electrodes 120, a control unit 130, and a processing unit 140. The headband 110 is worn on the head of a user. The surface of the headband has electrode mounting recesses. The electrodes 120 are mounted in the electrode mounting recesses. The electrodes 120 include at least one stimulation electrode 122 and at least one sensing electrode 124. The location of the stimulation electrode 122 on the headband corresponds to at least one brain region. The control unit 130 is configured to adjust the electrical stimulation parameter of the stimulation electrode 122 and the measurement parameter of the sensing electrode 124. The processing unit 140 is configured to obtain an electrical physiological signal from the sensing electrode 124.

In the embodiment, the electrode mounting recesses are located on the inner side or the outer side of the headband 110. In addition, the headband 110 may be made of a flexible material to guarantee wearing comfort and wearing fit, making it suitable for long-term use. The headband 110 may also include at least one positioning adjustment mechanism that ensures the accurate locations of stimulation and sensing electrodes based on anatomical landmarks of the user's head, such as the glabella, superior tragus, or inion, thereby enhancing treatment precision and effectiveness. The stimulation electrodes 122 and sensing electrodes 124 may be either dry or wet electrodes. The stimulation electrodes 122 and sensing electrodes 124 are made of biocompatible materials with good conductivity, such as silver/silver chloride (Ag/AgCl) or conductive silicone. The control unit 130 may adjust electrical stimulation parameters that include frequency, current intensity, pulse width, phase, and waveform according to a predefined treatment. Waveforms may include sine waves, triangular waves, and square waves, enabling effective stimulation of targeted brain regions.

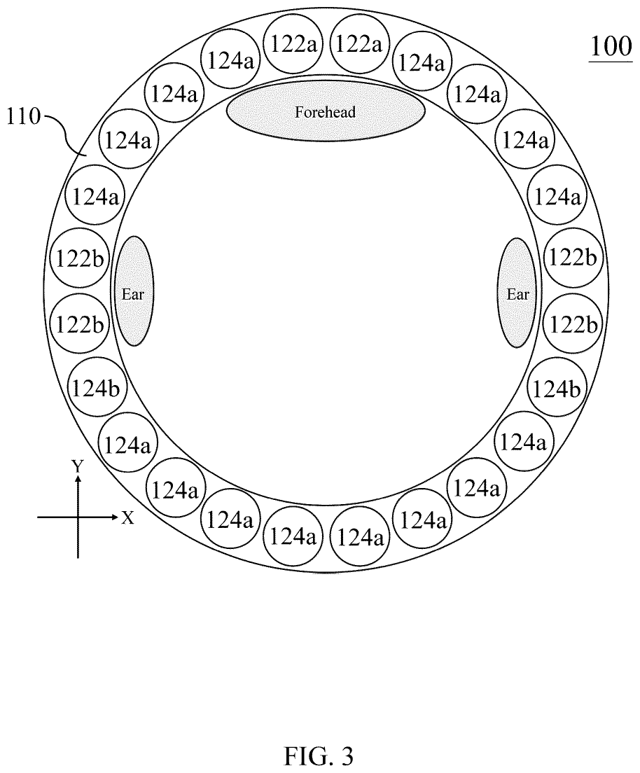

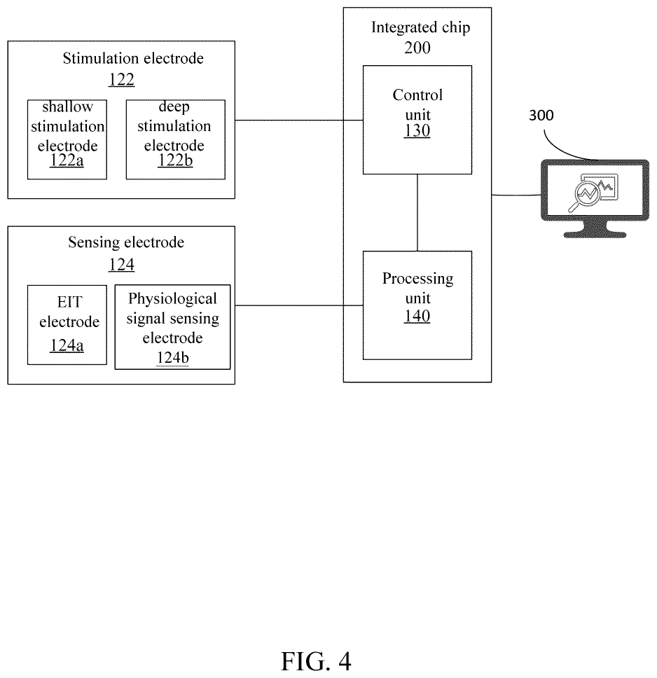

Please refer to FIGS. 3-4. FIG. 3 is a diagram schematically illustrating arrangement of the electrodes of a wearable neural stimulation and sensing device according to an embodiment of the invention. FIG. 4 is a diagram schematically illustrating a wearable neural stimulation and sensing device according to a second embodiment of the invention.

According to another embodiment, the stimulation electrodes 122 include shallow stimulation electrodes 122a and deep stimulation electrodes 122b. The shallow stimulation electrode 122a is configured to perform electrical stimulation of a shallow brain region, such as a cerebral cortical region. The deep stimulation electrodes 122b is configured to perform electrical stimulation of a deep brain region, such as basal ganglia, thalamus, hippocampus, or other deep structures.

In the embodiment, the shallow stimulation electrode 122a may be a trigeminal nerve stimulation (TNS) electrode. The deep stimulation electrode 122b may be configured as a temporal interference brain stimulation (TIBS) electrode and used to implement temporal interference brain stimulation (TIBS). FIG. 3 shows arrangement of electrodes for depression treatment. The locations of electrodes are described as follows.

The electrode mounting recess for the TNS electrode (i.e., the shallow stimulation electrode 122a) is located on the headband 110 corresponding to the user's forehead region. The TNS electrode directly stimulates the trigeminal nerve in the forehead. This enables effective stimulation of shallow brain regions, contributing to the alleviation of depressive symptoms.

The electrode mounting recess for the TIBS electrode (i.e., the deep stimulation electrode 122b) is located on the headband 110 corresponding to two opposite sides of the head of the user and located near the user's temporal lobes. The TIBS electrode performs non-invasive electrical stimulation of deep brain regions such as the subgenual cingulate gyrus (SCG) and the medial forebrain bundle (MFB) using temporal interference technology.

Furthermore, in one embodiment, the arrangement of electrodes shown in FIG. 3 is tailored for depression treatment. The wearable neural stimulation and sensing device 100 of the invention is not limited to the arrangement of electrodes. The electrode mounting recess in the device is highly flexible and adaptable according to different therapeutic requirements. All electrode mounting recesses are shareable. The locations of the stimulation and sensing electrodes are flexibly exchanged. For example, during temporal interference stimulation, the deep stimulation electrodes 122b may primarily serve as stimulation electrodes. However, in certain scenarios, the electrodes adjacent to the deep stimulation electrodes 122b may also be used for stimulation, thereby providing more flexible electrode arrangement to achieve optimal treatment outcomes. Therefore, the electrode arrangement can be adaptable according to the requirements and treatment goals of each patient. The invention is not limited to the specific arrangement illustrated in FIG. 3.

Additionally, the sensing electrodes 124 can be configured as electrical impedance tomography (EIT) electrodes (124a) or as electrical physiological signal sensing electrodes (124b). The electrical physiological signal sensing electrodes 124b are used for acquiring electroencephalogram (EEG), electrocardiography (ECG), photoplethysmography (PPG), or galvanic skin response (GSR) signals. The locations of the sensing electrodes 124 can adaptable according to the target parameters to be measured. For instance, in the embodiment shown in FIG. 3, in order to achieve optimal impedance imaging effect, the electrical impedance tomography electrodes 124a are distributed in most of the electrode mounting recesses on the headband 110 to form an electrode array surrounding the head. This arrangement improves the spatial resolution and imaging quality of impedance imaging, thereby providing accurate intracranial impedance distribution images.

Furthermore, in order to avoid signal interference between different types of electrical stimulation, the control unit 130 is configured to independently adjust the electrical stimulation parameters of the shallow stimulation electrodes 122a and the deep stimulation electrodes 122b, as well as the measurement parameters of the electrical impedance tomography electrodes 124a. These parameters include frequency, current stimulation and intensity, waveform, etc. The specific electrical measurement parameters for the embodiment are shown in Table 1:

TABLE 1
electrical stimulation and measurement parameters

| | Frequency | Current | | Interface | |
|---|---|---|---|---|---|
| Electrode | range | density | | impedance | |
| TNS electrode | 100 Hz~20 kHz | 0.1~14 | mA | 1~100 | kΩ |
| TIBS electrode | 2 kHz | 0.1~7 | mA | 5~20 | kΩ |
| | (<10 kHz) | | | | |
| EIT electrode | 10 kHz~100 kHz | 0.1~1.6 | mA | 0.1~10 | kΩ |

As shown in Table 1, regarding the electrical stimulation parameters of the TNS electrodes, test results indicate that a suitable stimulation current for shallow brain regions ranges from approximately 1 mA to 14 mA, with a stimulation frequency between 100 Hz and 20 KHz. Considering that the wearable neural stimulation and sensing device 100 is applied to various neurodegenerative diseases and based on human safety assessments, the stimulation current is set to range from 0.1 mA to 14 mA, and the stimulation frequency is set between 100 Hz and 20 KHz.

As shown in Table 1, regarding the electrical stimulation parameters of the TIBS electrodes, test results indicate that using 2 kHz and 2.005 kHz as stimulation frequencies and setting the current intensity between 0.5 to 3 mA successfully stimulated the hippocampus (i.e., a deep brain region). Since the depth of the affected brain regions of neurodegenerative diseases is similar to that of the hippocampus, a similar frequency range (e.g., 2 kHz) was selected as the carrier frequency for the main stimulation current in temporal interference brain stimulation. The current intensity for temporal interference brain stimulation is expanded to a range of 0.1 to 7 mA to accommodate therapeutic effects and various diseases. For patients with neurodegenerative diseases, factors such as the degree of lesion, neuronal damage, and disease type can affect the optimal stimulation current intensity. Therefore, a broader current range is designed to meet diverse clinical requirements. Based on safety regulations for temporal interference brain stimulation in humans, the upper limit of stimulation current is set at 7 mA to ensure safe treatment.

In another embodiment, the electrical physiological signal sensing electrodes 124b are preferably installed near the region behind the user's ears. Each electrical physiological signal sensing electrode 124b is installed behind each ear to effectively acquire the required electrical physiological signals. The region behind the ear, such as the region between the scalp and the earlobe, is suitable for sensing EEG, ECG, PPG, and GSR signals. Using appropriate amplifying and filtering circuits, the processing unit 140 processes and analyzes the sensed signals. The processing unit 140 features real-time data processing and analysis capabilities, and processes both electrical physiological signals and electrical impedance imaging data. Through a wireless communication module, the processing unit 140 can transmit results to the user or a medical personnel device 300 for therapeutic evaluation and parameter adjustment.

In summary, the embodiment in FIG. 3 not only ensures the installation of stimulation electrodes 122 at required stimulation regions but also enhances the imaging quality of EIT by adding more EIT electrodes 124a. Additionally, two electrical physiological signal sensing electrodes 124b are installed to acquire more electrical physiological signals, thereby aiding in the assessment of stimulation efficacy. By precisely controlling stimulation and measurement parameters, independence among various stimulation modes is maintained to avoid signal interference and enhance treatment effectiveness.

In yet another embodiment, the control unit 130 and the processing unit 140 can be integrated into a single integrated chip 200, as illustrated in FIG. 4. The integrated chip 200 includes various functional circuits to perform various device functions. The integrated chip 200 includes:
(1) Deep stimulation circuit that controls the deep stimulation electrodes 122b for temporal interference brain stimulation.
(2) Shallow stimulation circuit that controls the shallow stimulation electrodes 122a for trigeminal nerve stimulation.
(3) EIT readout circuit that processes signals from the EIT electrodes 124a to generate intracranial impedance distribution images.
(4) Electrical physiological signal readout circuit that processes signals from the electrical physiological sensing electrodes 124b, including EEG, ECG, PPG, and GSR.
(5) Low-power high-efficiency transceiver system circuit that enables wireless communication for transmitting processed data to external devices and receiving control commands.

During integration, signal transmission and hardware layout among different circuits require careful planning to optimize overall performance. Due to the different frequency ranges and current intensities involved in deep stimulation, shallow stimulation, EIT, and electrical physiological signals, functions such as frequency isolation and signal filtering are incorporated into the circuit design to prevent cross-interference. Specific parameters can be referenced in Table 1. Various circuit modules are arranged within the chip to reduce electromagnetic interference (EMI) and crosstalk. Isolation layers or shielding structures are added between critical modules to enhance signal integrity. The chip's size and shape are optimized for integration into the headband 110.

Furthermore, the integrated chip 200 can be fabricated using advanced microelectronics manufacturing technology such as CMOS (Complementary Metal-Oxide-Semiconductor) processes. Packaging can utilize System-in-Package (SiP) or System-on-Chip (SoC) technologies suitable for wearable devices to further reduce size and weight.

In practical applications, the integrated chip 200 can be installed at the suitable location of the headband 110 and connected to the stimulation electrodes 122 and the sensing electrodes 124 via wires or flexible printed circuit boards (FPCBs). To enhance wearing comfort and aesthetics, the chip and circuits may be wrapped within the structure or materials of the headband to avoid directly contacting skin.

Additionally, the integrated chip 200 is designed with modular architecture, leaving room for future functional expansion. This includes reserved interfaces for adding new sensing modules (e.g., body temperature and blood oxygen saturation sensors) or communication modules (e.g., 5G, NB-IoT), enhancing device adaptability and upgradeability.

Therefore, by integrating the control unit 130 and the processing unit 140 into the integrated chip 200, the embodiment achieves a highly integrated device with reduced size and weight, improved performance and reliability, and lower production costs. This makes the wearable neural stimulation and sensing device 100 more suitable for daily use, thereby providing patients with neurodegenerative diseases a portable and efficient tool for treatment and monitoring.

In further embodiment, to ensure that the stimulation electrodes consistently stimulate the same brain region throughout the stimulation process, multiple technical measures are implemented, including precise electrode positioning, current focusing technologies, high-precision control systems, and real-time physiological feedback mechanisms.

In the embodiment, the technical features for stimulating the same brain region include:

Precise electrode positioning: Stimulation electrodes 122 are precisely installed at the specific locations of the headband 110 corresponding to the anatomical locations of the target brain regions. Adjustable positioning mechanisms are included in the headband 110, using anatomical landmarks (e.g., glabella, external auditory canal, occipital protuberance) as reference points to ensure that the locations of the stimulation electrodes 122 are precisely aligned to the target brain region.

In conclusion, the embodiment of the invention fully adopts the advantages of electrical stimulation and EIT. The electrical design achieves precise stimulation, high integration, and portability. From a therapeutic perspective, using current as the stimulation source enables precise stimulation and high integration. Compared with magnetic or ultrasound-based stimulation methods, electrical stimulation directly modulates abnormal neuronal discharge. Moreover, electrical stimulation is easier to integrate stimulation, control, and readout circuits using standard microelectronic processes, thereby enabling single-chip integration without the need for specialized fabrication processes and enhancing systematic integration and reliability.

For measuring electrical impedance, the embodiment employs EIT to significantly reduce system size. Compared with conventional equipment that uses magnetic resonance imaging to obtain in vivo images, EIT is more convenient and easier to integrate with other systems. Its compactness and high level of integration help implement the lightweight and high-performance designs of wearable devices. Therefore, the device of the embodiment not only enhances the treatment effectiveness of neurodegenerative disease but also provides a more convenient and efficient therapeutic and monitoring solution for patients, thereby demonstrating wide application prospects.

The wearable neural stimulation and sensing device 100 of the embodiment enables targeted therapy for various neurodegenerative diseases using both temporal interference brain stimulation electrodes and trigeminal nerve stimulation electrodes. The wearable neural stimulation and sensing device 100 can provide effective treatment and symptom relief for neurodegenerative diseases such as Parkinson's disease, Alzheimer's disease, depression, schizophrenia, and epilepsy. Based on the pathological characteristics of different diseases, the device has targeted applications in the following aspects:

### 1. Parkinson's Disease:

Temporal interference brain stimulation is performed on temporal interference brain stimulation regions such as the subthalamic nucleus (STN) and internal globus pallidus (GPi) to help reduce tremors and rigidity.

Trigeminal nerve stimulation provides non-invasive pain relief, anxiety reduction, and alleviation of depressive symptoms.

Electrical physiological signal monitoring includes EEG for brainwave changes, ECG for heart rate variability, and PPG for cardiovascular assessment to evaluate treatment efficacy.

Unified Parkinson's Disease Rating Scale (UPDRS) as an evaluation index is used to track the symptom changes of patients and assess therapeutic effects.

### 2. Alzheimer's Disease:

Temporal interference stimulation is performed on temporal interference brain stimulation regions such as the nucleus basalis of Meynert (NBM) and fornix regions to promote the cognitive function of a patient.

Trigeminal nerve stimulation addresses depressive symptoms to alleviate psychiatric manifestations.

Electrical physiological signal monitoring tracks neural activities and emotional responses using electrical physiological signals such as EEG, ECG, and GSR.

Evaluation indexes include the Clinical Dementia Rating (CDR) and Alzheimer's Disease Assessment Scale-Cognitive Subscale (ADAS-Cog) that are used to assess the memory and the changes of cognitive performance of patients.

### 3. Depression:

Temporal interference stimulation is performed on temporal interference brain stimulation regions such as the subgenual cingulate gyrus (SCG) and medial forebrain bundle (MFB) of deep regions to improve abnormal brain discharges and relieve depressive symptoms.

Trigeminal nerve stimulation targets stress and anxiety of patients for immediate mood stabilization.

Electrical physiological signal monitoring employs EEG to monitor brainwave changes and employs PPG and GSR to detect the responses of an autonomic nervous system.

Hamilton Depression Rating Scale (HAM-D) as an evaluation index quantifies emotional changes.

### 4. Schizophrenia:

Temporal interference stimulation is performed on temporal interference brain stimulation regions such as the medial frontal cortex (MFC) and nucleus accumbens (NAcc) to reduce hallucinations and delusions.

Trigeminal nerve stimulation alleviates anxiety and social withdrawal, enhancing social functions.

Electrical physiological signal monitoring employs EEG to record brainwave changes and employs GSR and PPG to track the responses of autonomic nervous system.

The specific symptom rating scale as an evaluation index for schizophrenia is used to monitor patient conditions and assess efficacy.

### 5. Epilepsy:

Temporal interference stimulation is performed on temporal interference brain stimulation regions such as the anterior nucleus of the thalamus (ANT) and the hippocampus (HC) to reduce the frequency of epileptic seizures.

Trigeminal nerve stimulation alleviates muscle tension and fatigue following an epileptic seizure.

Electrical physiological signal monitoring employs EEG to monitor abnormal brain discharges and employs ECG and PPG to detect changes in cardiac function.

The long-term therapeutic effect is evaluated based on long-term brainwave monitoring and seizure frequency tracking as an evaluation index.

The treatment methods of the wearable neural stimulation and sensing device 100 for the foregoing diseases are summarized in Table 2.

TABLE 2
Treatment Regions, Electrical Physiological Signals, and Evaluation Indexes

| | | Trigeminal | | |
|---|---|---|---|---|
| | Temporal | Nerve | | |
| | Interference | Stimulation for | Electrical | |
| | Stimulation | Relieving | Physiological | Evaluation |
| Disease | Regions | Symptom | Signals | Indexes |
| Parkinson's | Subthalamic | Cognitive | EEG: | Tremor |
| Disease | Nucleus | Impairment | Movement | Severity |
| | (STN), | | Disorders | Basal |
| | Globus | | PPG: | Ganglia |
| | Pallidus | | Cardiovascular | Dopamine |
| | Internus | | Abnormalities | Level |
| | (GPi) | | | UPDRS |
| | | | | Rating Scale |
| Alzheimer's | Nucleus | Inhibiting | ECG: Heart | Brain |
| Disease | Basalis of | Neurodegeneration | Rate | atrophy |
| | Meynert | and | Variability | Clinical |
| | (NBM), | Inflammation | PPG: Heart | Dementia |
| | Fornix | | Rate | Rating |
| | | | Variability | (CDR) Scale |
| | | | GSR: | ADAS-Cog |
| | | | Sympathetic | Rating Scale |
| | | | Nerves | |
| | | | EEG: Sleep | |
| | | | Abnormalities | |
| Depression | Subgenual | Stress, Anxiety | EEG: | Brainwave |
| | Cingulate | | Emotional | Changes |
| | Gyrus | | Abnormalities | HAM-D |
| | (SCG), | | ECG: | Rating Scale |
| | Medial | | Emotional | |
| | Forebrain | | Abnormalities | |
| | Bundle | | PPG: | |
| | (MFB) | | Psychological | |
| | | | Stress | |
| | | | GSR: | |
| | | | Sympathetic | |
| | | | Nerves | |
| Schizophrenia | Medial | Anxiety, Social | EEG: | Selected |
| | Prefrontal | Withdrawal | Severity of | evaluation |
| | Cortex | | Illness | indexes |
| | (MFC), | | ECG: Heart | based on |
| | Nucleus | | Rate | symptoms, |
| | Accumbens | | Variability | such as |
| | (NAc) | | PPG: Heart | hallucinations, |
| | | | Rate | delusions, |
| | | | Variability | emotional |
| | | | | flattening |
| Epilepsy | Anterior | Muscle | EEG: | Brainwave |
| | Nucleus of | Tension, | Seizure | Changes |
| | Thalamus | Fatigue | Monitoring | Brain |
| | (ANT), | | ECG: | Images |
| | Hippocampus | | Seizure | |
| | (HC) | | Monitoring | |
| | | | PPG: | |
| | | | Seizure | |
| | | | Monitoring | |
| | | | GSR: | |
| | | | Seizure | |
| | | | Monitoring | |

The embodiments described above are only to exemplify the invention and not to limit the scope of the invention. Therefore, any equivalent modification or variation according to the shapes, structures, features, or spirit disclosed by the invention is to be also included within the scope of the invention.

What is claimed is:

1. A wearable neural stimulation and sensing device comprising:
a headband worn on a head of a user, wherein a surface of the headband has electrode mounting recesses;
a plurality of electrodes, mounted in the electrode mounting recesses, including at least one stimulation electrode and at least one sensing electrode, wherein a location of the at least one stimulation electrode on the headband corresponds to at least one brain region;
a control unit coupled to the at least one stimulation electrode and the at least one sensing electrode and configured to adjust an electrical stimulation parameter of the at least one stimulation electrode and a measurement parameter of the at least one sensing electrode; and
a processing unit coupled to the at least one sensing electrode and configured to obtain an electrical physiological signal from the at least one sensing electrode.

2. The wearable neural stimulation and sensing device according to claim 1, wherein the at least one stimulation electrode comprises:
a shallow stimulation electrode configured to perform electrical stimulation of a shallow brain region; and
a deep stimulation electrode configured to perform electrical stimulation of a deep brain region.

3. The wearable neural stimulation and sensing device according to claim 2, wherein the shallow stimulation electrode is a trigeminal nerve stimulation (TNS) electrode.

4. The wearable neural stimulation and sensing device according to claim 3, wherein the electrode mounting recesses are arranged on the headband corresponding to a forehead region of the head of the user and configured to mount the TNS electrode to stimulate a trigeminal nerve.

5. The wearable neural stimulation and sensing device according to claim 4, wherein the electrical stimulation parameter of the TNS electrode comprises a frequency range of 100 Hz˜20 kHz, a current intensity of 0.1˜14 mA, and an interface impedance of 1˜100 kΩ.

6. The wearable neural stimulation and sensing device according to claim 4, wherein the deep stimulation electrode is configured as a temporal interference brain stimulation (TIBS) electrode.

7. The wearable neural stimulation and sensing device according to claim 6, wherein the electrode mounting recesses are arranged on the headband corresponding to two opposite sides of the head of the user and configured to mount the TIBS electrode to stimulate the deep brain region.

8. The wearable neural stimulation and sensing device according to claim 7, wherein the electrical stimulation parameter of the TIBS electrode comprises a frequency range of less than 10 kHz, a current intensity of 0.1˜7 mA, and an interface impedance of 5˜20 kΩ.

9. The wearable neural stimulation and sensing device according to claim 7, wherein the deep brain region is a subcallosal cingulate gyrus (SCG) or a medial forebrain bundle (MFB).

10. The wearable neural stimulation and sensing device according to claim 1, wherein the sensing electrode is configured as an electrical impedance tomography (EIT) electrode, and the processing unit is coupled to the sensing electrode and configured to obtain an intracranial impedance distribution from the EIT electrode to generate an intracranial impedance distribution image.

11. The wearable neural stimulation and sensing device according to claim 10, wherein the measurement parameter of the EIT electrode comprises a frequency range of 10 kHz˜100 kHz, a current intensity of 0.1˜1.6 mA, and an interface impedance of 0.1˜10 Ω.

12. The wearable neural stimulation and sensing device according to claim 1, wherein the at least one sensing electrode is configured as an electrical physiological signal sensing electrode, arranged near a postauricular region of the user, and configured to sense electrical physiological signals related to one or a combination of a heart, blood vessels, and skin, and the processing unit is coupled to an electrical physiological signal sensor and configured to obtain electrical physiological signals, thereby generating a corresponding electrocardiogram (ECG), photoplethysmogram (PPG), or galvanic skin response (GSR).

13. A wearable neural stimulation device comprising:
a headband worn on a head of a user, wherein a surface of the headband has electrode mounting recesses;
a plurality of electrodes, mounted in the electrode mounting recesses, including at least two stimulation electrodes, wherein the at least two stimulation electrodes are positioned on the headband at locations corresponding to at least one brain region, and the at least two stimulation electrodes include a trigeminal nerve stimulation (TNS) electrode and a temporal interference brain stimulation (TIBS) electrode; and
a control unit configured to adjust electrical stimulation parameters of the at least two stimulation electrodes.

14. The wearable neural stimulation device according to claim 13, wherein the electrical stimulation parameter of the TNS electrode comprises a frequency range of 100 Hz˜20 kHz, a current intensity of 0.1˜14 mA, and an interface impedance of 1˜100 kΩ.

15. The wearable neural stimulation device according to claim 13, wherein the electrical stimulation parameter of the TIBS electrode comprises a frequency range of less than 10 kHz, a current intensity of 0.1˜7 mA, and an interface impedance of 5˜20 kΩ.
